SEQUENCE LISTING (8 sequences)

SEQ ID NO: 1 - protein, 659 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 2 - DNA, 2611 bp, Homo sapiens
aactgagtggctgtgaaagggtggggtttgctcagactgtccttcctctctggactgtaagaatatgtctccagggccagtgtctgctgcgatcgagtcccaccttccaagtcctggcatctcaatgcatctgggaagctacctgcattaagtcaggactgagcacacaggtgaactccagaaagaagaagctatggccgcagtgattctggagagcatctttctgaagcgatcccaacagaaaaagaaaacatcacctctaaacttcaagaagcgcctgtttctcttgaccgtgcacaaactctcctactatgagtatgactttgaacgtgggagaagaggcagtaagaagggttcaatagatgttgagaagatcacttgtgttgaaacagtggttcctgaaaaaaatcctcctccagaaagacagattccgagaagaggtgaagagtccagtgaaatggagcaaatttcaatcattgaaaggttcccttatcccttccaggttgtatatgatgaagggcctctctacgtcttctccccaactgaagaactaaggaagcggtggattcaccagctcaaaaacgtaatccggtacaacagtgatctggttcagaaatatcacccttgcttctggatcgatgggcagtatctctgctgctctcagacagccaaaaatgctatgggctgccaaattttggagaacaggaatggaagcttaaaacctgggagttctcaccggaagacaaaaaagcctcttcccccaacgcctgaggaggaccagatcttgaaaaagccactaccgcctgagccagcagcagcaccagtctccacaagtgagctgaaaaaggttgtggccctttatgattacatgccaatgaatgcaaatgatctacagctgcggaagggtgatgaatattttatcttggaggaaagcaacttaccatggtggagagcacgagataaaaatgggcaggaaggctacattcctagtaactatgtcactgaagcagaagactccatagaaatgtatgagtggtattccaaacacatgactcggagtcaggctgagcaactgctaaagcaagaggggaaagaaggaggtttcattgtcagagactccagcaaagctggcaaatatacagtgtctgtgtttgctaaatccacaggggaccctcaaggggtgatacgtcattatgttgtgtgttccacacctcagagccagtattacctggctgagaagcaccttttcagcaccatccctgagctcattaactaccatcagcacaactctgcaggactcatatccaggctcaaatatccagtgtctcaacaaaacaagaatgcaccttccactgcaggcctgggatacggatcatgggaaattgatccaaaggacctgaccttcttgaaggagctggggactggacaatttggggtagtgaagtatgggaaatggagaggccagtacgacgtggccatcaagatgatcaaagaaggctccatgtctgaagatgaattcattgaagaagccaaagtcatgatgaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctggcagctcgaaactgtttggtaaacgatcaaggagttgttaaagtatctgatttcggcctgtccaggtatgtcctggatgatgaatacacaagctcagtaggctccaaatttccagtccggtggtccccaccggaagtcctgatgtatagcaagttcagcagcaaatctgacatttgggcttttggggttttgatgtgggaaatttactccctggggaagatgccatatgagagatttactaacagtgagactgctgaacacattgcccaaggcctacgtctctacaggcctcatctggcttcagagaaggtatataccatcatgtacagttgctggcatgagaaagcagatgagcgtcccactttcaaaattcttctgagcaatattctagatgtcatggatgaagaatcctgagctcgccaataagcttcttggttctacttctcttctccacaagccccaatttcactttctcagaggaaatcccaagcttaggagccctggagcctttgtgctcccactcaatacaaaaaggcccctctctacatctgggaatgcacctcttctttgattccctgggatagtggcttctgagcaaaggccaagaaattattgtgcctgaaatttcccgagagaattaagacagactgaatttgcgatgaaaatattttttaggagggaggatgtaaatagccgcacaaaggggtccaacagctctttgagtaggcatttggtagagcttgggggtgtgtgtgtgggggtggaccgaatttggcaagaatgaaatggtgtcataaagatgggaggggagggtgttttgataaaataaaattactagaaagcttgaaagtc

SEQ ID NO: 3 - DNA, 217 bp, Homo sapiens
gaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctg

SEQ ID NO: 4 - DNA, 20 bp, Artificial Sequence
  Synthetic construct
cagttgtatggcgtctgcac

SEQ ID NO: 5 - DNA, 20 bp, Artificial Sequence
  Synthetic construct
tccaggtattccatggcttc

SEQ ID NO: 6 - DNA, 14 bp, Artificial Sequence
  Synthetic construct
aatggctgcctcct

SEQ ID NO: 7 - DNA, 14 bp, Artificial Sequence
  Synthetic construct
aatggctccctcct

SEQ ID NO: 8 - DNA, 14 bp, Artificial Sequence
  Synthetic construct
aatggcagcctcct

(19) United States
(12) Patent Application Publication
(10) Pub. No.: US 2018/0208993 A1
(43) Pub. Date: Jul. 26, 2018

(54) HIGHLY SENSITIVE METHODS FOR DETECTING BTK RESISTANCE MUTATIONS IN RNA AND DNA

(71) Applicant: OHIO STATE INNOVATION FOUNDATION, Columbus, OH (US)

(72) Inventors: Daniel Jones, Columbus, OH (US); Huolin Tu, Lewis Center, OH (US); John Byrd, Columbus, OH (US)

(21) Appl. No.: 15/816,738

(22) Filed: Nov. 17, 2017

Related U.S. Application Data
(60) Provisional application No. 62/423,531, filed on Nov. 17, 2016.
(60) Provisional application No. 62/526,736, filed on Jun. 29, 2017.

(51) Int. Cl. C12Q 1/6886

(52) U.S. Cl. CPC C12Q 1/6886; C12Q 2600/106; C12Q 2600/156

(57) ABSTRACT

Disclosed is a highly sensitive mutation-specific quantitative polymerase chain reaction (PCR) assay to detect BTK mutations in B cells.

### CROSS REFERENCE TO RELATED APPLICATIONS

This application claims the benefit of U.S. Provisional Patent Application Ser. No. 62/423,531, filed Nov. 17, 2016, and U.S. Provisional Patent Application Ser. No. 62/526,736, filed Jun. 29, 2017, each of which is expressly incorporated herein by reference in its entirety.

### STATEMENT OF GOVERNMENT SUPPORT

This work was supported by R01 CA197870, R01 CA183444, R01 CA177292, K23 CA178183, P50 CA140158, P01 CA095426, and R35 CA197734 awarded by the National Cancer Institutes. The government has certain rights in the invention.

### BACKGROUND

The Bruton agammaglobulinemia tyrosine kinase (BTK) activates B-cell receptor signaling through activation of phospholipase C gamma 2 (PLCG2). Clinical resistance to Bruton tyrosine kinase (BTK) inhibitors such as ibrutinib in chronic lymphocytic leukemia (CLL) is highly associated with emergence of the BTK C481 mutations that prevents ibrutinib covalent binding. Emergence of the BTK C481 mutated CLL, beginning 23 years after therapy initiation, has been shown to correlate with large cell transformation and poor outcome. The ability to properly identify people CLL having a BTKL C481 mutation enables treatment regimens to be adjusted to employ alternatives not affected by the mutation. Accordingly, what are needed methods and kits for detecting BTK inhibitor resistance.

Most of the mutations involve the C481S amino acid change (produced by 3 different nucleotide changes) but may also C481R, C481F, C481V changes. This diversity of these mutations has complicated understanding of development of resistance, including whether both BTK genes within a single cell need to be concurrently mutated and whether the mutations arise independently.

### SUMMARY

Disclosed is a highly sensitive mutation-specific quantitative polymerase chain reaction (PCR) assay to detect BTK mutations in B cells. Products are analyzed using digital droplet (d) PCR (RainDrop platform, Raindance Technologies) that allows amplification and counting of single molecules. ddPCR has the advantage over conventional PCR of detecting the base sequence of a single DNA molecule using a twostep process amplification and detection process. This allows very accurate and highly quantitative count of the number of mutated nucleic acid species in a mixed cell population. It also measurement of the mutation profile of single cells by dilution.

The assay uses separate fluorescently-labeled probes for the wildtype sequence and any BTK mutations/variants (e.g. c.1442G>C and c.1441T>A) that are not exon spanning permitting their use in with either DNA or RNA (cDNA). This allows a determination of the mutation/wildtype ratio independent in RNA and DNA obtained from any given biologic sample. This allows determination of both the percentage of mutated cells in the sample and the level of overexpression or underexpression of mutated BTK molecules.

Dilution of cells to achieve single cells for direct DNA isolation allow the mechanistic studies on the development of multiple BTK C481 mutation species. Both of the data sets are difficult to achieve using other mutation detection techniques.

Comparison of mutated DNA/normal DNA from DNA and RNA simultaneously on the same sample allows assessment of whether the level of ibrutinib resistance can be explained by differential expression of mutated and wildtype BTK transcripts.

The details of one or more embodiments of the invention are set forth in the accompanying drawings and the description below. Other features, objects, and advantages of the invention will be apparent from the description and drawings, and from the claims.

In one aspect, disclosed herein are methods for detecting a Bruton agammaglobulinemia tyrosine kinase (BTK) mutation in B cells, comprising assaying DNA and RNA in B cells for one or more BTK mutations by polymerase chain reaction PCR, comparing the number of DNA and RNA mutations in each cell to identify a mutation:wildtype ratio, thereby determining both the percentage of mutated cells in the sample and the level of expression of the mutated BTK molecules in each cell.

Also disclosed herein are methods for detecting resistance to a BTK inhibitor in a subject with chronic lymphocytic leukemia (CLL), comprising assaying DNA and RNA in B cells from the subject for one or more BTK mutations by PCR, comparing the number of DNA and RNA mutations in each cell to identify a mutation:wildtype ratio, wherein an increase in the percentage of cells comprising a BTK mutation in the sample, an increase in the level of expression of the mutated BTK molecules in one or more cells, or a combination thereof indicates that the subject has a cancer that is resistant to BTK inhibitors.

In one aspect, disclosed herein are methods of treating chronic lymphocytic leukemia (CLL) comprising assaying nucleic acid in B cells from the subject for one or more BTK mutations by PCR, comparing the number of DNA and RNA mutations in each cell to identify a mutation:wildtype ratio, wherein an increase in the percentage of cells comprising a BTK mutation in the sample, an increase in the level of expression of the mutated BTK molecules in one or more cells, or a combination thereof indicates that the subject has a cancer that is resistant to BTK inhibitors; wherein in a subject with a BTK inhibitor resistant cancer the method further comprises administering to the subject a cancer therapeutic that is not a BTK inhibitor; and wherein in a subject without a BTK inhibitor resistant cancer the method comprises administering to the subject a BTK inhibitor.

Also disclosed are methods of any preceding aspect, wherein the PCR reaction is selected from the group consisting of digital droplet PCR (ddPCR), digital droplet qualitative PCR (ddQPCR), reverse transcriptase PCR (RT-PCR), and real-time PCR.

Also disclosed are methods of any preceding aspect, wherein the RNA is assayed by converting it first to cDNA using a reverse transcriptase.

Also disclosed are methods of any preceding aspect, wherein the BTK mutation comprises a mutation at C481 (for example, C481S, C481R, C481F, and/or C481V) as set forth in SEQ ID NO: 1.

Also disclosed are methods of any preceding aspect, wherein the BTK mutation comprises one or more mutation at G1442 or T1441 (for example T1441A, T1441C, T1441G, G1442C, G1442, and/or G1442A) as set forth in SEQ ID NO: 2.

Also disclosed are methods of any preceding aspect, wherein the BTK inhibitor comprises Ibrutinib, Acalabrutinib, ONO/GS-4059, Spebrutnib, HM71224, or BGB-3111.

### DESCRIPTION OF DRAWINGS

FIG. 1 shows the ddPCR patterns for the two detected BTK C481S mutations (MUT1: c.1442G>C; MUT2 c.1441T>A), BTK C481 WT and NTC.

FIG. 2 shows the cumulative incidence of ibrutinib discontinuation for transformation or chronic lymphocytic leukemia (CLL) progression. Discontinuation as a result of Richter transformation tended to occur early in ibrutinib therapy with few transformations after 2 years. In contrast, CLL progression occurred later, with rare progressions occurring before 1 year. Other events, which include infection, other toxicity, comorbidities, and patient or physician choice, tended to occur at a fairly steady rate for the first 3 years and then plateau.

FIG. 3 shows the survival after ibrutinib discontinuation. Patients with disease progression while on treatment with ibrutinib were observed from the time of ibrutinib discontinuation until death. Patients who discontinued therapy as a result of transformation had a median survival of 3.9 months, and patients who discontinued therapy as a result of progressive chronic lymphocytic leukemia (CLL) had a median survival of 22.7 months.

FIG. 4 shows that Ibrutinib resistance mutations can be detected before clinical relapse. For 20 patients with a detectable mutation in BTK or PLCG2 at time of relapse, samples before relapse were analyzed retrospectively to determine the interval of time between mutation detection and clinical relapse. An initial clone could be detected at an estimated median of 9.3 months before relapse. UPN, unique patient number.

FIG. 5 shows the sequential monitoring of BTK mutation levels. BTK/PLCG2 sequencing was performed every 3 months using the clinical-grade sequencing assay. In the eight patients with BTKC481S detected at greater than 1% variant allelic frequency (VAF) who had not experienced clinical relapse, VAF did increase in all patients.

### DETAILED DESCRIPTION

Chronic lymphocytic leukemia (CLL) is a leukemia primarily affecting white blood cells, in particular B cells. The disease starts in the bone marrow and quickly spreads to other cells. Treatment for CLL includes radiation therapy, chemotherapy, stem cell transplant and/or immunotherapy. Recent therapies have include the use of kinase inhibitors, such as, Bruton agammaglobulinemia tyrosine kinase (BTK) inhibitors (such as, for example, Ibrutinib, Acalabrutinib, ONO/GS-4059, Spebrutnib, HM71224, and/or BGB-3111) which block B cell receptor signaling.

The Bruton agammaglobulinemia tyrosine kinase (BTK) activates B-cell receptor signaling through activation of phospholipase C gamma 2 (PLCG2). Blocking this pathway inhibits proliferation of the cancerous cell and promotes apoptosis. However, clinical resistance to Bruton tyrosine kinase (BTK) inhibitors such as Ibrutinib in chronic lymphocytic leukemia (CLL) has been identified.

Resistance to BTK inhibitors is highly associated with emergence of the BTK mutations resulting in a substitution of the cysteine at residue 481 of BTK as set forth in SEQ ID NO: 1 that prevent ibrutinib covalent binding. Emergence of the BTK C481 mutated CLL, beginning 23 years after therapy initiation, has been shown to correlate with large cell transformation and poor outcome. Thus, the ability to properly identify people CLL having a BTK C481 mutation enables treatment regimens to be adjusted to employ alternatives not affected by the mutation. Accordingly, in one aspect, disclosed herein are methods for detecting a Bruton agammaglobulinemia tyrosine kinase (BTK) mutation (for example a substitution at residue 481 as shown in SEQ ID NO: 3) in B cells. In one aspect, it is understood and herein contemplated that the disclosed mutations in BTK confer resistance to BTK inhibitors which are commonly used to treat CLL. Thus, it is important to be able to detect BTK inhibitor resistant cancers in subject. Accordingly, in one aspect, disclosed herein are methods of detecting resistance to a BTK inhibitor in a subject with chronic lymphocytic leukemia (CLL). It is further understood that the detection of a mutation in BTK that confers BTK inhibitor resistance (for example, the amino acid substitution at residue 481 of SEQ ID NO: 1) is important for prescribing a subject with CLL the appropriate treatment as a BTK inhibitor should not be administered to a subject with CLL if said subject has a BTK inhibitor resistant CLL. Thus, in one aspect, disclosed herein are also methods of treating CLL, comprising amongst other things detecting a Bruton agammaglobulinemia tyrosine kinase (BTK) mutation and thereby resistance to a BTK inhibitor.

Substitutions at amino acid residue 481 of BTK as set forth in SEQ ID NO: 1, can comprise cysteine to serine (C481S) substitutions, cysteine to arginine (C481R) substitutions, cysteine to phenylalanine (C481F) substitutions, and cysteine to valine (C481V) substitutions. These amino acid substitutions can be the result of one nucleic acid substitutions of the nucleic acid of BTK as set forth in SEQ ID NO: 2. In particular, the nucleic acid substitution can be one or more nucleic acid substitutions at residues 1441 or 1442 (residues 1634 and 1635, respectively of SEQ ID NO: 2 which has a coding start at residue 194). For example, a C481S amino acid substitution can be encoded by a T1441A or G1442C nucleic acid substitution, a C481R amino acid substitution can be encoded by a T1441C nucleic acid substitution, a C481F amino acid substitution can be encoded by a G1442T nucleic acid substitution, and a C421V amino acid substation can be encoded by a T1441G and G1442T nucleic acid substitution.

The methods disclosed herein relate to the detection of nucleic acid or amino acid variation in the form of, for example, point mutations of BTK (such as, for example, an amino acid substitution at residue C481 (as set forth in SEQ ID NO: 1) and/or a substitution of a nucleic acid at residue T1441 and/or G1442 as set forth in SEQ ID NO: 2. In one aspect, it is understood and herein contemplated that the disclosed methods of detecting a mutation in BTK can be applied to detect resistance to BTK inhibitors. Accordingly, in one aspect, disclosed herein are methods for detecting resistance to a BTK inhibitor in a subject with chronic lymphocytic leukemia (CLL) comprising detecting the presence of a nucleic acid and/or amino acid variation that confers said resistance (for example detection of a nucleic acid substitution at residues 1441 and/or 1442 as set forth in SEQ ID NO: 2 and/or detection of an amino acid substitution at residue 481 as set forth in SEQ ID NO: 1.

For these latter expression level detections, the methods comprise detecting either the abundance or presence of RNA, DNA, cDNA, amino acids, and/or mRNA. Thus, disclosed herein are methods for detecting a BTK mutation in B cells, methods of detecting resistance to a BTK inhibitor in a subject with CLL, or methods of treating CLL comprising amongst other things detecting a BTK inhibitor resistance mutation (e.g., a C481 and/or a G1442 substitution, such as C481S, C481R, C481F, C481V, G1442C, and G1442A), comprising measuring the presence of one or more BTK mutations in the nucleic acid (DNA. RNA, cDNA, and/or mRNA) in each cell from a tissue sample from the subject; and comparing the frequency and level of mutation relative to noncancerous (i.e., wildtype) cells.

A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, reverse transcription-polymerase chain reaction (RT-PCR), real-time PCR, real-time RT-PCR, digital droplet PCR (ddPCR), digital droplet quantitative PCR (ddQPCR), and microarray. Therefore, also disclosed herein are methods for detecting a BTK mutation in B cells, methods of detecting resistance to a BTK inhibitor in a subject with CLL, and/or methods of treating CLL comprising conducting a nucleic acid amplification process on a tissue sample from the subject and detecting the presence of mutations in the nucleic acid associated with BTK in the tissue sample, wherein the nucleic acid amplification process is ddPCR.

In theory, each of these techniques can be used to detect specific RNAs and to precisely determine their expression level. In general, Northern analysis is the only method that provides information about transcript size, whereas NPAs are the easiest way to simultaneously examine multiple messages. In situ hybridization is used to localize expression of a particular gene within a tissue or cell type, and RT-PCR is the most sensitive method for detecting and quantitating gene expression.

RT-PCR allows for the detection of the RNA transcript of any gene, regardless of the scarcity of the starting material or relative abundance of the specific mRNA. In RT-PCR, an RNA template is copied into a complementary DNA (cDNA) using a retroviral reverse transcriptase. The cDNA is then amplified exponentially by PCR using a DNA polymerase. The reverse transcription and PCR reactions can occur in the same or difference tubes. RT-PCR is somewhat tolerant of degraded RNA. As long as the RNA is intact within the region spanned by the primers, the target will be amplified.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) often vary in expression and, therefore, may not be appropriate internal controls. Additionally, most common internal controls are expressed at much higher levels than the mRNA being studied. For relative RT-PCR results to be meaningful, all products of the PCR reaction must be analyzed in the linear range of amplification. This becomes difficult for transcripts of widely different levels of abundance.

Competitive RT-PCR is used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

Digital droplet PCR and ddQPCR utilize nanoliter reaction sizes. Unlike quantitative PCR reactions, ddPCR and DDQPCR can measure the actual number of molecules (target DNA) as each molecule is in one droplet as opposed to flurosence intensity as in QPCR, thus making it a discrete “digital” measurement. It provides absolute quantification because dPCR measures the positive fraction of samples, which is the number of droplets that are fluorescing due to proper amplification. This positive fraction accurately indicates the initial amount of template nucleic acid.

Thus, disclosed herein in one aspect are methods for detecting a BTK mutation in B cells, methods of detecting resistance to a BTK inhibitor in a subject with CLL, or methods of treating CLL comprising conducting a nucleic acid amplification process on a tissue sample from the subject comprising conducting an ddPCR or DDQPCR reaction on mRNA from a tissue sample from the subject; wherein the assay comprises reverse transcription of RNA to cDNA using a reverse transcriptase with one or more primers capable of specifically hybridizing to one or more BTK sequences and at least one forward primer capable of specifically hybridizing to one or more BTK kinase sequences; and comparing the frequency and level of mutation relative to noncancerous (i.e., wildtype) cells wherein an increase in the ratio of mutated cells to non-mutated cells (i.e., wildtype) or the presence of mutation at residue 481 or 1442 of BTK indicates a BTK mutation, resistance to one or more BTK inhibitors, and that treatment regimens should be adjusted to stop usage of a BTK inhibitor and use any of the accepted alternative treatment strategies for CLL.

While the disclosed methods can include pCR based assays, the methods disclosed herein are not limited to such methods and can include any other nucleic acid based detection method.

Northern analysis is the easiest method for determining transcript size, and for identifying alternatively spliced transcripts and multigene family members. It can also be used to directly compare the relative abundance of a given message between all the samples on a blot. The Northern blotting procedure is straightforward and provides opportunities to evaluate progress at various points (e.g., intactness of the RNA sample and how efficiently it has transferred to the membrane). RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

The Nuclease Protection Assay (NPA) (including both ribonuclease protection assays and S1 nuclease assays) is a sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. Solution hybridization is typically more efficient than membrane-based hybridization, and it can accommodate up to 100 μg of sample RNA, compared with the 20-30 μg maximum of blot hybridizations. NPAs are also less sensitive to RNA sample degradation than Northern analysis since cleavage is only detected in the region of overlap with the probe (probes are usually about 100-400 bases in length).

NPAs are the method of choice for the simultaneous detection of several RNA species. During solution hybridization and subsequent analysis, individual probe/target interactions are completely independent of one another. Thus, several RNA targets and appropriate controls can be assayed simultaneously (up to twelve have been used in the same reaction), provided that the individual probes are of different lengths. NPAs are also commonly used to precisely map mRNA termini and intron/exon junctions.

In situ hybridization (ISH) is a powerful and versatile tool for the localization of specific mRNAs in cells or tissues. Unlike Northern analysis and nuclease protection assays, ISH does not require the isolation or electrophoretic separation of RNA. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents.

DNA Detection and Quantification

As indicated throughout, the methods disclosed herein relate to the detection of nucleic acid variation in the form of, for example, point mutations of expression of BTK. For these latter expression level detections, the methods comprise detecting either the abundance or presence of mRNA, or both. Alternatively, detection can directed to the abundance or presence of DNA, for example, cDNA. Thus, disclosed herein are methods for detecting a mutation at residue 481 of BTK; methods of detecting resistance to a BTK inhibitor in a subject with a cancer (such as, for example CLL) comprising assaying DNA and RNA in B cells for one or more BTK mutations by digital droplet polymerase chain reaction (ddPCR), comparing the number of DNA and RNA mutations in each cell to identify a mutation:wildtype ratio, thereby determining both the percentage of mutated cells in the sample and the level of expression of the mutated BTK molecules in each cell. It is understood and wherein contemplated that methods of detecting resistance to a BTK inhibitor in a subject with a cancer, an increase in the percentage of cells comprising a BTK mutation in the sample, an increase in the level of expression of the mutated BTK molecules in one or more cells, or a combination thereof indicates that the subject has a cancer that is resistant to BTK inhibitors. It is understood and herein contemplated that the primers used in the pCR reaction can be a forward and reverse primer pair that can specifically hybridize to BTK. It is also understood that the disclosed methods can be performed and measured relative to a negative control.

A number of widely used procedures exist for detecting and determining the abundance of a particular DNA in a sample. For example, the technology of PCR permits amplification and subsequent detection of minute quantities of a target nucleic acid. Details of PCR are well described in the art, including, for example, U.S. Pat. No. 4,683,195 to Mullis et al., U.S. Pat. No. 4,683,202 to Mullis and U.S. Pat. No. 4,965,188 to Mullis et al. Generally, oligonucleotide primers are annealed to the denatured strands of a target nucleic acid, and primer extension products are formed by the polymerization of deoxynucleoside triphosphates by a polymerase. A typical PCR method involves repetitive cycles of template nucleic acid denaturation, primer annealing and extension of the annealed primers by the action of a thermostable polymerase. The process results in exponential amplification of the target nucleic acid, and thus allows the detection of targets existing in very low concentrations in a sample. It is understood and herein contemplated that there are variant PCR methods known in the art that may also be utilized in the disclosed methods, for example, Quantitative PCR (QPCR); microarrays, real-time PCT; hot start PCR; nested PCR; allele-specific PCR; digital droplet PCR (ddPCR), digital droplet quantitative PCR (ddQPCR), and Touchdown PCR.

Microarrays

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GeneChip® (Affymetrix, Inc which refers to its high density, oligonucleotide-based DNA arrays), and gene array.

DNA microarrays, or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein contemplated that the disclosed microarrays can be used to monitor gene expression, disease diagnosis, gene discovery, drug discovery (pharmacogenomics), and toxicological research or toxicogenomics.

There are two variants of the DNA microarray technology, in terms of the property of arrayed DNA sequence with known identity. Type I microarrays comprise a probe cDNA (500˜5,000 bases long) that is immobilized to a solid surface such as glass using robot spotting and exposed to a set of targets either separately or in a mixture. This method is traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

To prepare beads coated with immobilized probes, beads are immersed in a solution containing the desired probe sequence and then immobilized on the beads by covalent or noncovalent means. Alternatively, when the probes are immobilized on rods, a given probe can be spotted at defined regions of the rod. Typical dispensers include a micropipette delivering solution to the substrate with a robotic system to control the position of the micropipette with respect to the substrate. There can be a multiplicity of dispensers so that reagents can be delivered to the reaction regions simultaneously. In one embodiment, a microarray is formed by using ink-jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate.

Tissue samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. In one embodiment, total RNA is isolated using the TRIzol total RNA isolation reagent (Life Technologies, Inc., Rockville, Md.) and RNA is isolated using oligo d(T) column chromatography or glass beads. After hybridization and processing, the hybridization signals obtained should reflect accurately the amounts of control target polynucleotide added to the sample.

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one embodiment, biotin is first incorporated during an amplification step as described above. After the hybridization reaction, unbound nucleic acids are rinsed away so that the only biotin remaining bound to the substrate is that attached to target polynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art.

Methods for detecting complex formation are well known to those skilled in the art. In one embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In one embodiment, individual polynucleotide probe/target complex hybridization intensities are normalized using the intensities derived from internal normalization controls contained on each microarray.

Type II microarrays comprise an array of oligonucleotides (20˜80-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its photolithographically fabricated products under the GeneChip® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz, and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules, attached to the silane matrix, provide a surface that may be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4 nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

In one aspect, disclosed herein are microarrays wherein an oligonucleotide probe in use on the microarray specifically hybridizes to a BTK. In one aspect, disclosed herein are methods of detecting a Bruton agammaglobulinemia tyrosine kinase (BTK) mutation in B cells, comprising assaying DNA and RNA in B cells for one or more BTK mutations by microarray or oligonucleotide probe, comparing the number of DNA and RNA mutations in each cell to identify a mutation:wildtype ratio, thereby determining both the percentage of mutated cells in the sample and the level of expression of the mutated BTK molecules in each cell. Also disclosed are methods of detecting a BTK mutation and methods of detecting a BTK inhibitor resistant cancer in a subject comprising measuring the amount of DNA and RNA in B cells for one or more BTK mutations; wherein the BTK wildtype probe comprises /5 TET/AAT+G̅G̅C̅T̅+G̅C̅/ZEN/+C̅T̅+C̅C̅T̅/3IABkFQ/ (SEQ ID NO: 6, underlined portion only); wherein the BTK Mut Probe 1 (c.1442G>C) comprises /56 FAM/A̅A̅T̅+G̅G̅C̅T̅+C̅C̅/ZEN/+C̅T̅+C̅C̅T̅/3IABkFQ/ (SEQ ID NO: 7, underlined portion only); and wherein the BTK Mut Probe 2 (c.1441T>A) comprises /56 FAM/A̅A̅T̅+G̅G̅C̅+A̅G̅C̅/ZEN/+C̅T̅+C̅C̅T̅/3IABkFQ/ (SEQ ID NO: 8, underlined portion only).

Real-Time PCR

Real-time PCR monitors the fluorescence emitted during the reaction as an indicator of amplicon production during each PCR cycle (i.e., in real time) as opposed to the endpoint detection. The real-time progress of the reaction can be viewed in some systems. Real-time PCR does not detect the size of the amplicon and thus does not allow the differentiation between DNA and cDNA amplification, however, it is not influenced by non-specific amplification unless SYBR Green is used. Real-time PCR quantitation eliminates post-PCR processing of PCR products. This helps to increase throughput and reduce the chances of carryover contamination. Real-time PCR also offers a wide dynamic range of up to 10⁷-fold. Dynamic range of any assay determines how much target concentration can vary and still be quantified. A wide dynamic range means that a wide range of ratios of target and normaliser can be assayed with equal sensitivity and specificity. It follows that the broader the dynamic range, the more accurate the quantitation. When combined with RT-PCR, a real-time RT-PCR reaction reduces the time needed for measuring the amount of amplicon by providing for the visualization of the amplicon as the amplification process is progressing.

The real-time PCR system is based on the detection and quantitation of a fluorescent reporter. This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles can indicate the detection of accumulated PCR product.

A fixed fluorescence threshold is set significantly above the baseline that can be altered by the operator. The parameter C_{T} (threshold cycle) is defined as the cycle number at which the fluorescence emission exceeds the fixed threshold.

There are three main fluorescence-monitoring systems for DNA amplification: (1) hydrolysis probes; (2) hybridising probes; and (3) DNA-binding agents. Hydrolysis probes include TaqMan probes, molecular beacons and scorpions. They use the fluorogenic 5′ exonuclease activity of Taq polymerase to measure the amount of target sequences in cDNA samples.

TaqMan probes are oligonucleotides longer than the primers (20-30 bases long with a Tm value of 10° C. higher) that contain a fluorescent dye usually on the 5′ base, and a quenching dye (usually TAMRA) typically on the 3′ base. When irradiated, the excited fluorescent dye transfers energy to the nearby quenching dye molecule rather than fluorescing (this is called FRET=Förster or fluorescence resonance energy transfer). Thus, the close proximity of the reporter and quencher prevents emission of any fluorescence while the probe is intact. TaqMan probes are designed to anneal to an internal region of a PCR product. When the polymerase replicates a template on which a TaqMan probe is bound, its 5′ exonuclease activity cleaves the probe. This ends the activity of quencher (no FRET) and the reporter dye starts to emit fluorescence which increases in each cycle proportional to the rate of probe cleavage. Accumulation of PCR products is detected by monitoring the increase in fluorescence of the reporter dye (note that primers are not labelled). TaqMan assay uses universal thermal cycling parameters and PCR reaction conditions. Because the cleavage occurs only if the probe hybridises to the target, the origin of the detected fluorescence is specific amplification. The process of hybridisation and cleavage does not interfere with the exponential accumulation of the product. One specific requirement for fluorogenic probes is that there be no G at the 5′ end. A ‘G’ adjacent to the reporter dye can quench reporter fluorescence even after cleavage.

Molecular beacons also contain fluorescent (FAM, TAMRA, TET, ROX) and quenching dyes (typically DABCYL) at either end but they are designed to adopt a hairpin structure while free in solution to bring the fluorescent dye and the quencher in close proximity for FRET to occur. They have two arms with complementary sequences that form a very stable hybrid or stem. The close proximity of the reporter and the quencher in this hairpin configuration suppresses reporter fluorescence. When the beacon hybridises to the target during the annealing step, the reporter dye is separated from the quencher and the reporter fluoresces (FRET does not occur). Molecular beacons remain intact during PCR and must rebind to target every cycle for fluorescence emission. This will correlate to the amount of PCR product available. All real-time PCR chemistries allow detection of multiple DNA species (multiplexing) by designing each probe/beacon with a spectrally unique fluor/quench pair as long as the platform is suitable for melting curve analysis if SYBR green is used. By multiplexing, the target(s) and endogenous control can be amplified in single tube.

With Scorpion probes, sequence-specific priming and PCR product detection is achieved using a single oligonucleotide. The Scorpion probe maintains a stem-loop configuration in the unhybridised state. The fluorophore is attached to the 5′ end and is quenched by a moiety coupled to the 3′ end. The 3′ portion of the stem also contains sequence that is complementary to the extension product of the primer. This sequence is linked to the 5′ end of a specific primer via a non-amplifiable monomer. After extension of the Scorpion primer, the specific probe sequence is able to bind to its complement within the extended amplicon thus opening up the hairpin loop. This prevents the fluorescence from being quenched and a signal is observed.

Another alternative is the double-stranded DNA binding dye chemistry, which quantitates the amplicon production (including non-specific amplification and primer-dimer complex) by the use of a non-sequence specific fluorescent intercalating agent (SYBR-green I or ethidium bromide). It does not bind to ssDNA. SYBR green is a fluorogenic minor groove binding dye that exhibits little fluorescence when in solution but emits a strong fluorescent signal upon binding to double-stranded DNA. Disadvantages of SYBR green-based real-time PCR include the requirement for extensive optimisation. Furthermore, non-specific amplifications require follow-up assays (melting point curve or dissociation analysis) for amplicon identification. The method has been used in HFE-C282Y genotyping. Another controllable problem is that longer amplicons create a stronger signal (if combined with other factors, this may cause CDC camera saturation, see below). Normally SYBR green is used in singleplex reactions, however when coupled with melting point analysis, it can be used for multiplex reactions.

The threshold cycle or the C_{T} value is the cycle at which a significant increase in ΔRn is first detected (for definition of ΔRn, see below). The threshold cycle is when the system begins to detect the increase in the signal associated with an exponential growth of PCR product during the log-linear phase. This phase provides the most useful information about the reaction (certainly more important than the end-point). The slope of the log-linear phase is a reflection of the amplification efficiency. The efficiency (Eff) of the reaction can be calculated by the formula: Eff=10^{(-1/slope)}−1. The efficiency of the PCR should be 90-100% (3.6>slope>3.1). A number of variables can affect the efficiency of the PCR. These factors include length of the amplicon, secondary structure and primer quality. Although valid data can be obtained that fall outside of the efficiency range, the qRT-PCR should be further optimised or alternative amplicons designed. For the slope to be an indicator of real amplification (rather than signal drift), there has to be an inflection point. This is the point on the growth curve when the log-linear phase begins. It also represents the greatest rate of change along the growth curve. (Signal drift is characterised by gradual increase or decrease in fluorescence without amplification of the product.) The important parameter for quantitation is the C_{T}. The higher the initial amount of genomic DNA, the sooner accumulated product is detected in the PCR process, and the lower the C_{T} value. The threshold should be placed above any baseline activity and within the exponential increase phase (which looks linear in the log transformation). Some software allows determination of the cycle threshold (C_{T}) by a mathematical analysis of the growth curve. This provides better run-to-run reproducibility. A C_{T} value of 40 means no amplification and this value cannot be included in the calculations. Besides being used for quantitation, the C_{T} value can be used for qualitative analysis as a pass/fail measure.

Multiplex TaqMan assays can be performed using multiple dyes with distinct emission wavelengths. Available dyes for this purpose are FAM, TET, VIC and JOE (the most expensive). TAMRA is reserved as the quencher on the probe and ROX as the passive reference. For best results, the combination of FAM (target) and VIC (endogenous control) is recommended (they have the largest difference in emission maximum) whereas JOE and VIC should not be combined. It is important that if the dye layer has not been chosen correctly, the machine will still read the other dye's spectrum. For example, both VIC and FAM emit fluorescence in a similar range to each other and when doing a single dye, the wells should be labelled correctly. In the case of multiplexing, the spectral compensation for the post run analysis should be turned on (on ABI 7700: Instrument/Diagnostics/Advanced Options/Miscellaneous). Activating spectral compensation improves dye spectral resolution.

Nested PCR

The disclosed methods can further utilize nested PCR. Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction. Nested PCR is often more successful in specifically amplifying long DNA fragments than conventional PCR, but it requires more detailed knowledge of the target sequences.

Thus, disclosed herein in one aspect are methods of detecting a Bruton agammaglobulinemia tyrosine kinase (BTK) mutation in B cells, comprising assaying DNA and RNA in B cells for one or more BTK mutations; methods of detecting resistance to a BTK inhibitor in a subject with chronic lymphocytic leukemia (CLL), comprising assaying DNA and RNA in B cells from the subject for one or more BTK mutations; and methods of treating a cancer comprising a BTK inhibitor resistance mutation the method comprising amongst other things assaying DNA and RNA in B cells from the subject for one or more BTK mutations wherein the PCR reaction (for example, ddPCR) comprises a reverse primer capable of specifically hybridizing to one or more BTK sequences (for example, TCCAGGTATTCCATGGCTTC (SEQ ID NO: 5)) and at least one forward primer capable of specifically hybridizing to one or more BTK sequences (for example, CAGTTGTATGGCGTCTGCAC (SEQ ID NO: 4)).

### Primers and Probes

As used herein, “primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

As used herein, “probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Disclosed are compositions including primers and probes, which are capable of interacting with the disclosed BTK nucleic acids such as SEQ ID NO: 2 or its complement. In certain embodiments the primers are used to support nucleic acid extension reactions, nucleic acid replication reactions, and/or nucleic acid amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are disclosed. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids. As an example of the use of primers, one or more primers can be used to create extension products from and templated by a first nucleic acid.

The size of the primers or probes for interaction with the nucleic acids can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the nucleic acid of interest typically will be used to produce extension products and/or other replicated or amplified products that contain a region of the nucleic acid of interest. The size of the product can be such that the size can be accurately determined to within 3, or 2 or 1 nucleotides.

In certain embodiments the product can be, for example, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product can be, for example, less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

Thus, it is understood and herein contemplated that the disclosed RT-PCR and PCR reactions that comprise a portion of the disclosed methods or performed using the disclosed kits require forward and reverse primers to form a primer pair. Herein disclosed, the forward primer can be a primer that specifically hybridizes to an BTK such as, for example, SEQ ID NO: 4. The reverse primer can also comprise a primer that specifically hybridizes to BTK such as, for example, SEQ ID NO: 5. Thus, in one aspect, disclosed herein are kits and methods wherein the forward primer is SEQ ID NO: 4 and the reverse primer is SEQ ID NO: 5. For example, in one aspect, disclosed herein are methods of detecting a Bruton agammaglobulinemia tyrosine kinase (BTK) mutation in B cells, method for detecting resistance to a BTK inhibitor in a subject with chronic lymphocytic leukemia (CLL), and methods of treating chronic lymphocytic leukemia (CLL) comprising in a subject; the methods comprising assaying DNA and RNA in B cells from the subject for one or more BTK mutations by PCR reaction (for example, by digital droplet polymerase chain reaction (ddPCR)), and comparing the number of DNA and RNA mutations in each cell to identify a mutation:wildtype ratio, wherein the PCR reaction comprises at least one forward primer and at least one reverse primer that specifically hybridizes to a BTK nucleic acid as set forth in SEQ ID NO: 2 (such as, for example SEQ ID NO: 4 and SEQ ID NO: 5).

It is understood and herein contemplated that there are situations where it may be advantageous to utilize more than one primer pair to detect the presence of a fusion, truncation, or over expression mutation. Such RT-PCR or PCR reactions can be conducted separately, or in a single reaction. When multiple primer pairs are placed into a single reaction, this is referred to as “multiplex PCR.” For example, the reaction can comprise a first BTK forward and reverse primer pair, as well as, second BTK forward and reverse primer (for example SEQ ID NO: 4 and SEQ ID NO: 5, respectively). In some instances, the second forward and reverse primer can be internal (i.e., nested) to the first BTK forward and reverse primer.

Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends of the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers and scorpion primers.

Cleavage activated primers are similar to cleavage activated probes except that they are primers that are incorporated into replicated strands and are then subsequently cleaved.

Labels

To aid in detection and quantitation of nucleic acids produced using the disclosed methods, labels can be directly incorporated into nucleotides and nucleic acids or can be coupled to detection molecules such as probes and primers. As used herein, a label is any molecule that can be associated with a nucleotide or nucleic acid, directly or indirectly, and which results in a measurable, detectable signal, either directly or indirectly. Many such labels for incorporation into nucleotides and nucleic acids or coupling to nucleic acid probes are known to those of skill in the art. Examples of labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, CASCADE BLUE®, OREGON GREEN®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy Fl, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow SGF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Phycoerythrin B, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

The absorption and emission maxima, respectively, for some of these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037 which is incorporated herein by reference.

Labeled nucleotides are a form of label that can be directly incorporated into the amplification products during synthesis. Examples of labels that can be incorporated into amplified nucleic acids include nucleotide analogs such as BrdUrd, aminoallyldeoxyuridine, 5-methylcytosine, bromouridine, and nucleotides modified with biotin or with suitable haptens such as digoxygenin. Suitable fluorescence-labeled nucleotides are Fluorescein-isothiocyanate-dUTP, Cyanine-3-dUTP and Cyanine-5-dUTP. One example of a nucleotide analog label for DNA is BrdUrd (bromodeoxyuridine, BrdUrd, BrdU, BUdR, Sigma-Aldrich Co). Other examples of nucleotide analogs for incorporation of label into DNA are AA-dUTP (aminoallyldeoxyuridine triphosphate, Sigma-Aldrich Co.), and 5-methyl-dCTP (Roche Molecular Biochemicals). One example of a nucleotide analog for incorporation of label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these labels are also considered labels. Any of the known labels can be used with the disclosed probes, tags, and method to label and detect nucleic acid amplified using the disclosed method. Methods for detecting and measuring signals generated by labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary label coupled to the antibody. As used herein, detection molecules are molecules which interact with amplified nucleic acid and to which one or more labels are coupled.

The disclosed methods can be used to detect a mutation in BTK in any disease where uncontrolled cellular proliferation occurs herein referred to as “cancer”. A non-limiting list of different types of BTK related cancers is as follows: lymphomas (Hodgkins and non-Hodgkins), leukemias, carcinomas, carcinomas of solid tissues, chronic lymphocyte leukemia, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed methods can be used to diagnose is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Thus, disclosed herein are methods of diagnosing a cancer wherein the cancer is selected from the group consisting of non-small cell lung carcinoma, diffuse large B-cell lymphoma, chronic lymphocytic leukemia (CLL), systemic histiocytosis, breast cancer, colorectal carcinoma, esophageal squamous cell carcinoma, anaplastic large-cell lymphoma, neuroblastoma, and inflammatory myofibroblastic tumors (IMTs). For example, disclosed herein are methods of diagnosing CLL in a subject comprising assaying DNA and/or RNA in B cells from the subject for one or more BTK mutations by PCR (for example, digital droplet polymerase chain reaction (ddPCR), ddQPCR, RT-PCR, and/or real-time PCR), comparing the number of DNA and RNA mutations in each cell to identify a mutation:wildtype ratio, wherein an increase in the percentage of cells comprising a BTK mutation in the sample, an increase in the level of expression of the mutated BTK molecules in one or more cells, or a combination thereof indicates that the subject has a cancer that is resistant to BTK inhibitors; wherein in a subject with a BTK inhibitor resistant cancer the method further comprises administering to the subject a cancer therapeutic that is not a BTK inhibitor and wherein in a subject without a BTK inhibitor resistant cancer the method comprises administering to the subject a BTK inhibitor (such as, for example, Ibrutinib, Acalabrutinib, ONO/GS-4059, Spebrutnib, HM71224, and/or BGB-3111).

The disclosed methods of detecting a Bruton agammaglobulinemia tyrosine kinase (BTK) mutation in B cells, method for detecting resistance to a BTK inhibitor in a subject with chronic lymphocytic leukemia (CLL), and methods of treating chronic lymphocytic leukemia (CLL), assay DNA, cDNA, RNA, mRNA, amino acids, peptides, polypeptides, and/or proteins obtained from one or more tissue samples from a subject. In one aspect the tissue sample can be obtained from any tissue from which nucleic acids, proteins, peptides, or amino acids may be obtained, including, but not limited to whole blood, peripheral blood mononuclear cells (PBMC), serum, saliva, bone marrow, tissue lavage, and biopsy (including but not limited to core biopsy).

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: BTK Resistance Mutation (C481S)

Bruton tyrosine kinase (BTK) inhibitors, such as ibrutinib, are widely used in the treatment of chronic lymphocytic leukemia (CLL) and other B-cell lymphoproliferative disorders. Prolonged treatment with BTK inhibitors can result in the emergence of mutations in BTK, particularly C481S, that are associated with disease levels of BTK C481S can herald eventual disease progression and consideration of therapy switch. This assay sensitively detects the C481S mutation in BTK (c.1442G>C or c.1441T>A) using mutation-specific digital droplet PCR. BTK C481S mutations have not been detected in CLL patients prior to treatment with BTK inhibitors. First detection of BTK C481S mutation is most common after 2-4 years of ibrutinib treatment.

To detect a BTK mutation in a tissue sample from a subject, a PCR reaction can be performed on the DNA. cDNA, mRNA, RNA, proteins, peptides, and/or polypeptides from the subject. In one aspect, the PCR reaction can be a quantitative mutation/allele-specific digital droplet polymerase chain reaction (AS-ddPCR). The methods disclosed herein can detect a single point mutation in nucleic acids that encode a single point mutation in the amino acid sequence of BTK, this substitution conferring resistance to a BTK inhibitor. For example, the Ibrutinib therapy-resistance mutation BTK C481S BTK_Exon 15 (217 bp)/ENSE00000673980 GAATCTTTCCCATGAGAAGCTGGTGCAGTTGTATGGCGTCTGCACCAAGCAGCGCCC CATCTTCATCATCACTGAGTACATGGCCAATGGCTGCCTCCTGAACTACCTGAGGGA GATGCGCCACCGCTTCCAGACTCAGCAGCTGCTAGAGATGTGCAAGGATGTCTGTG AAGCCATGGAATACCTGGAGTCAAAGCAGTTCCTTCACCGAGACCTG (SEQ ID NO: 3).

Probes and primers for detecting the C481 mutation can include the primers and probes set forth in Table 1.

TABLE 1
Probe location; C481;
forward and reverse primers

| Probe Type | PRIMETIME LNA-ZEN (IDT) |
|---|---|
| BTK Primer_F | CAGTTGTATGGCGTCTGCAC |
| | (SEQ ID NO: 4) |
| BTK Primer_R | TCCAGGTATTCCATGGCTTC |
| | (SEQ ID NO: 5) |
| BTK WT Probe | /5TET/AAT + GGCT + GC/ |
| | ZEN/ + CT + CCT/3IABkFQ/ |
| | (SEQ ID NO: 6) |
| BTK Mut Probe 1 | /56FAM/AAT + GGCT + CC/ |
| (c.1442G > C) | ZEN/ + CT + CCT/3IABkFQ/ |
| | (SEQ ID NO: 7) |
| BTK Mut Probe 2 | /56FAM/AAT + GGC + AGC/ |
| (c.1441T > A) | ZEN/ + CT + CCT/3IABkFQ/ |
| | (SEQ ID NO: 8) |
| Amplicon Length | 164 bp |

The disclosed methods can comprise a BTK gene that is amplified using digital droplet PCR with fluorochrome-labeled probes for BTK wild-type sequence (TET-labeled) and separately BTK C481S c.1442G>C, BTK C481S c.1441T>A, BTK C481R c. 1441T>C; BTK C481F c. 1442G>T, or BTK C481V c. 1441T>G and 1442G>T mutant probes (FAM-labeled), on the Raindrop digital droplet PCR platform. The BTK C481mut (for example C481S, C481R, C481F, or C481V)/wild-type ratio is calculated by counting droplet positive for each signal.

Each reaction contains: 25 ul 2× TaqMan Genotyping Master Mix, 2 ul 25× Droplet Stablizer, 5 ul BTK primer F (5 uM), 5 ul BTK primer R (5 uM), 2.5 ul BTK WT probe (5 uM), 2.5 ul BTK Mut probe 1 (5 uM), 2.5 ul BTK Mut probe 2 (5 uM), 5.5 ul DNA (30 ng-200 ng), Note: PCR Mixes and Aliquots Should Be Done on Ice,

For Droplet PCR, the Thermal cycler Program can include:

Step 1: 95° C. for 10 minutes

Step 2: 95° C. for 15 seconds

Step 3: *60° C. for 15 seconds

Step 4: 60° C. for 45 seconds

Step 5: Go to step 2 to 4 and repeat for 44 more cycles

Step 6: 98° C. for 10 minutes

Step 7: 12° C. forever
a slow ramp rate (0.5 C/sec) is recommended from denaturation to annealing

Possible results of the PCR of a C481S mutation reaction include:

1) the BTK C481S mutation is NOT detected by digital droplet PCR in DNA extracted from leukocytes;

2) the BTK C481S mutation associated with resistance to BTK inhibitors is detected by digital droplet PCR in DNA extracted from leukocytes. The level of mutation is expressed as a ratio of BTK mutant events to total events in DNA extracted from total leukocytes. It will differ from the percentage calculated from B-cell preparations in the prior BTK-PLCG2 sequencing assay;

3) the BTK C481S mutation associated with resistance to BTK inhibitors is detected by digital droplet PCR in DNA extracted from leukocytes. The level of mutation is expressed as a ratio of BTK mutant events to total events. The level is increased compared to a prior result;

4) the BTK C481S mutation associated with resistance to BTK inhibitors is detected by digital droplet PCR in DNA extracted from leukocytes. The level of mutation is expressed as a ratio of BTK mutant events to total events. The mutant level is decreased compared to a prior result.

5) the BTK C481S mutation is NOT detected by digital droplet PCR in DNA extracted from immunodensity-purified B-cells; and

6) the BTK C481S mutation associated with resistance to BTK inhibitors is detected by digital droplet PCR in DNA extracted from immunodensity-purified B-cells. The level of mutation is expressed as a ratio of BTK mutant events to total events.

A quantitative polymerase chain reaction (PCR) assay was used to detect BTK C481S mutations using fluorescently-labeled probes for the wild-type sequence and the mutant BTK C481S sequences. Mutations besides BTK C481S (c.1441T>A and c.1442G>C) will not be detected by this assay. The sensitivity of detection is approximately 0.1% mutation-bearing cells in the analyzed sample; samples with signal below that cutoff will be reported as negative. Serial monitoring may be useful for assessing the clinical significance of low levels of BTK mutation level or the presence of mutation in minimally involved CLL samples.

These tests were developed and their performance characteristics were determined by the Polaris Molecular Laboratories of the Ohio State University Comprehensive Cancer Center, 2001 Polaris Parkway, Columbus, Ohio Performance characteristics refer to the analytical performance of the test. They have not been cleared by the US Food and Drug Administration (FDA). The FDA has determined that such clearance or approval is not necessary.

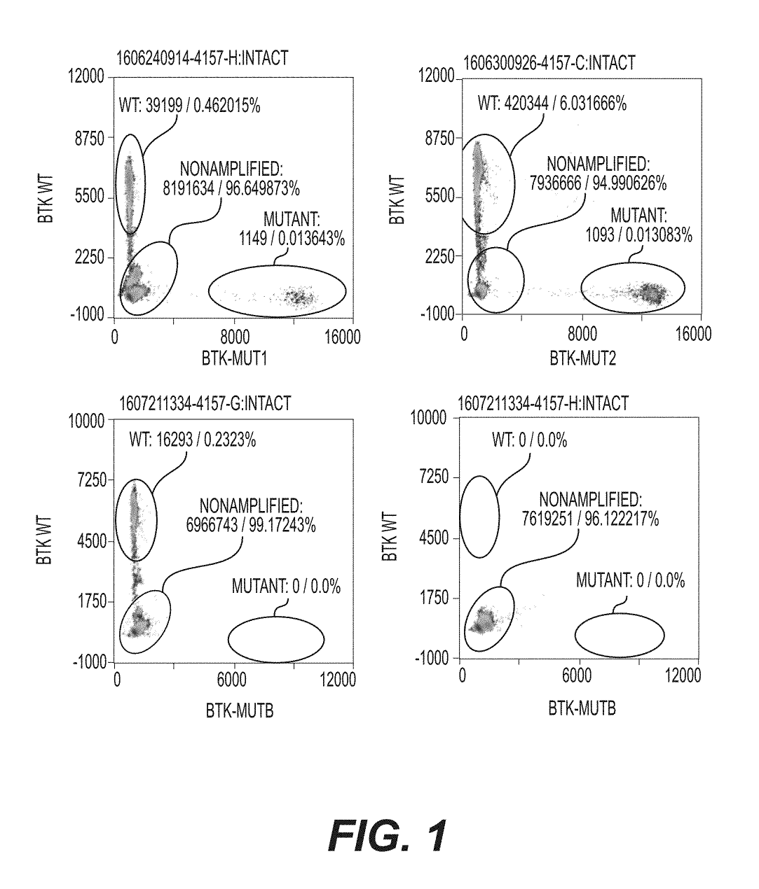

FIG. 1 shows the ddPCR patterns for the two detected BTK C481S mutations (MUT1: c.1442G>C; MUT2 c.1441T>A), BTK C481 WT and NTC.

Prior results, if available, can be used for comparison when a mutation is identified. Overall results of case including morphological and immunophenotypic features are taken into consideration for every case.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### Example 2: BTKC481S-Mediated Resistance to Ibrutinib in Chronic Lymphocytic Leukemia

Methods

Patients

All patients at The Ohio State University (OSU) Comprehensive Cancer Center (Columbus, Ohio) who were enrolled onto four Institutional Review Board-approved sequential trials of ibrutinib in patients with CLL were included in this analysis. OSU 10032/Pharmacyclics (PCYC) 1102 (ClinicalTrials.gov identifier: NCT01105247) was a multi-institutional phase IB/II study of single-agent ibrutinib in patients with relapsed, refractory, or treatment-naive CLL. OSU 10053/PCYC 1109 (NCT01217749) was a single-institution phase II study of ibrutinib in combination with ofatumumab in patients with relapsed or refractory CLL. OSU 11133 (NCT01589302) was a single-institution phase II study of single-agent ibrutinib in patients with relapsed or refractory CLL. OSU 12024/RESONATE (NCT01578707) was a multi-institutional phase III study of single-agent ibrutinib versus ofatumumab in patients with relapsed or refractory CLL. From this study, all patients initially assigned to ibrutinib or who crossed over to ibrutinib after progression with ofatumumab were included at that time.

Targeted Deep Sequencing

BTK and PLCG2 were analyzed with the use of the Ion Torrent Platform from Life Technologies (Carlsbad, Calif.).

Statistical Analysis

Time to discontinuation of ibrutinib therapy was measured from the first date of treatment until the off-study date, censoring patients who had not discontinued ibrutinib therapy at the date of last contact; patients who went off study for transplantation (n=5) or who continued treatment elsewhere (n=9) were censored at that time. Fine and Gray models of cumulative incidence were fit using forward selection to identify variables most strongly associated with discontinuing ibrutinib therapy as a result of transformation or CLL progression. Discontinuation of therapy for other adverse reasons, such as severe toxicity, was accounted for in the models as a competing risk. Variables considered for model inclusion were age, sex, number of prior therapies, baseline lactate dehydrogenase level, fluorescent in situ hybridization abnormalities [del(17)(p13.1), del(11)(q22.3), trisomy 12, or del(13)(q14)], MYC abnormalities, BCL6 abnormalities, complex karyotype ($ three abnormalities), and IGHV mutational status. All models adjusted for monotherapy with ibrutinib versus combination therapy with ibrutinib and ofatumumab, regardless of statistical significance. When the failure type was CLL progression, potentially important prognostic variables failed to meet the proportional hazards assumption as a result of few events occurring early in the study. For this reason, a landmark analysis at 1 year was performed, including only patients who remained on ibrutinib therapy at that time (n=247); only two CLL progressions had occurred before the landmark time and were excluded from the analysis. Because the hazard ratios from the landmark analysis better represent the risk of CLL progression during the time frame when progressions occurred and because the final proportional hazards models when using all data or a subset of the data in the landmark analysis included the same variables, only results from the landmark analysis are reported.

In the subgroups of patients who discontinued therapy as a result of transformation or CLL progression, survival was calculated from the off-study date until the date of death from any cause, censoring patients at last contact. Survival estimates were calculated using the Kaplan-Meier method, and differences between subgroups were tested using the log-rank test. In the subgroup of patients who experienced relapse with BTK or PLCG2 mutations and had serial samples available before relapse, a Kaplan-Meier estimate of the median time to first detection from date of relapse was obtained using interval censoring because a clone of resistant cells was known to develop between two sample submission dates.

A two-sided α=0.05 was used to declare statistical significance. All statistical analyses were performed using either TIBCO Spotfire S+ Version 8.2 (TIBCO Software, Palo Alto, Calif.) or SAS statistical software Version 9.4 (SAS Institute, Cary, N.C.).

Ion Torrent Deep Sequencing

Ion Torrent deep sequencing was carried out either in the research laboratory setting or the clinical laboratory setting with mild variation in methods.

Clinical Laboratory Methods:

B-lymphocytes were isolated from peripheral blood collected in EDTA-containing tubes using magnetic beads (RosetteSep Human B Cells, StemCell Technologies, Vancouver, BC, Canada). Samples containing at least 5×104 CD19+ B-cells, based on parallel flow cytometry studies, were generally required for adequate DNA recovery. Mutation analysis was performed by a custom-designed 87-amplicon Ion sequencing assay that covered the entire coding regions of the PLCG2 and BTK. Total genomic DNA was isolated from the B-cells using the QIAamp DNA Micro Kit (Qiagen, Chatsworth, Calif.). The concentration of the DNA was determined using the Qubit 2.0 Fluorometer (ThermoFisher). The extracted DNA was amplified, barcoded (Ion Xpress™ Barcode Adapters 1-16 Kit), and adapter ligated (Ion AmpliSeg™ Library Kit 2.0), followed by emulsion PCR 9 (Ion PGM™ Hi-Q™ Chef Kit or Ion PGM™ Template OT2 200 Kit) and sequencing with the Ion Personal Genome Machine (PGM) using Ion PGM™ Sequencing 200 Kit v2 chemistry. Sixteen patient samples or controls were analyzed per run (Ion 318 Chip Kit v2). All sequence reagents were from Life Technologies (Carlsbad, Calif.).

The sequencing data was analyzed by the Torrent Suite Software version 4.0.2 and mutation calling initiated by the Variant Caller plug-in (version 4.0-r76860). Variant calls were also independently evaluated by the GenomOncology platform and the variant calls merged. All genetic alterations were subject to molecular pathologist review prior to reporting. The mutations were reported according to Human Genome Variation Society (HGVS) guidelines.

Depth of coverage in the assay was biased for the BTK C481 hotspot with a mean coverage of 3013 reads (range 336-7691) per sample covering this site. Based on dilution studies, the lower limit of accurate quantitation for the C481 hotspot was established as approximately 1% mutation-bearing alleles for C481S c.1442G>C, with reproducible qualitative detection in the 0.1% range. Similar sensitivity was established for the C481S c.1441T>A and C481F (c.1442G>) transversions. Based on dilution studies, the assay was slightly less sensitive (2-5%) for the less common C481R and C481Y transition mutations as well as the multinucleotide C481S mutation (c.1442_1443delGCinsCT). Non-hotspot BTK and PLCG2 sites were validated at a 4% sensitivity based on bioinformatic criteria. Across a range of runs, the mean number of amplicons with depth of coverage greater than 250 reads per sample was 84.5 of 87 amplicons.

Research Laboratory Methods:

DNA was extracted from cryopreserved isolated CLL cells using DNA extraction kit (QIAamp DNA Mini Kit from Qiagen) according to manufacturer recommendations. DNA was quantified using spectrophotometric method (Nanodrop 2000 from Thermo Scientific) using standard 260/280 OD ratio. Analysis of the BTK and PLC-gamma genes was performed using next generation sequencing Ion Torrent platform and reagents from Life Technologies (Carlsbad, Calif.). Library was prepared with Ion AmpliSeq Library kit2.0 (4475345) with custom designed panel of AmpliSeq primers (panel design IAD48992, pipe line version 3.0, 87 amplicons in 2 pools, 17 kb panel size, 99.68% coverage) that covers the entire coding sequence and intronic splice acceptor and donor sites for both genes and IonExpress barcode adapters (kit#4471250 and #4474009). DNA was amplified on GeneAmp PCR system 9700 Dual 96-well thermal cycler from Applied Biosystems. PCR product was purified with Agencourt AMPure XP kit (A63881 Beckman Coulter, Indianapolis, Ind.). Library was quantified using real time PCR with Ion Library TAQMAN Quantitation kit 44688022 on (Applied Biosystems ViiA7 Real Time PCR System) instrument to allow for optimal final dilution of library for template preparation on OneTouch OT2 version instrument with Ion PGM Template OT2 200Kit (4480974). The ISPs enrichment and purification was performed on Ion One Touch2 ES. Purified ISPs were analyzed on Ion Torrent personal Genome Machine using IonPGM Sequencing 200v2 kit (4482006) and 318 chips v2 (4484354). Data were collected and analyzed using Torrent Server (4462616) with Torrent Suite 4.6 version. Final analysis of sequence data was performed using a combination of software: Variant Caller v.4.6.-11, IGV3.6.033 and Ion Reporter v.4.6. The following reference sequences were used for analysis; for BTK NM000061.2 and for PLCG2 NM002661.3. The entire length of sequences was reviewed manually using these programs to assess for deviation from reference sequence and to evaluate the quality of sequence and the depth of coverage. The depth of coverage ranged from 1000 to 15000 for all amplicons.

Results

Rate of CLL Progression on Ibrutinib Increases with Extended Follow-Up

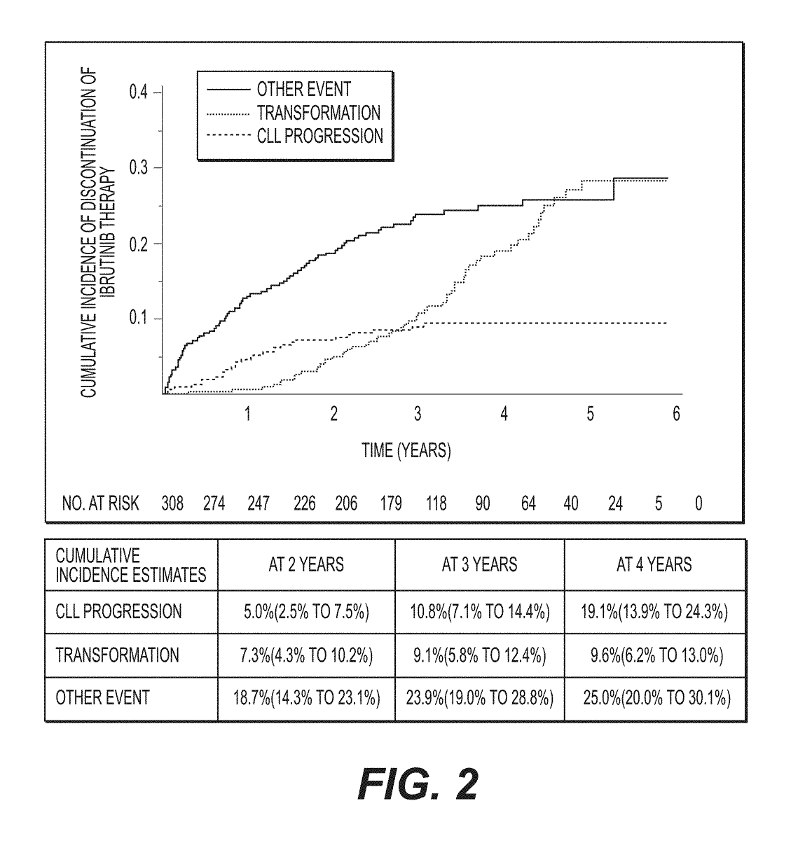

All 308 patients treated with ibrutinib on four sequential clinical trials at OSU were included in this analysis, with data locked as of Jun. 14, 2016. This includes 237 patients treated with single-agent ibrutinib on three clinical trials and 71 patients treated with ibrutinib in combination with ofatumumab. Patients had high-risk genetic features; 80% had IGHV unmutated disease, 58% had complex stimulated karyotype, and 40% had del(17)(p13.1). Eight patients were previously untreated, and all of these patients were age 65 years or older. Overall, patients had a median of three prior therapies (range, zero to 16 therapies). With a median follow-up time of 3.4 years (range, 0.3 to 5.9 years), 136 patients (44%) remain on therapy, 14 patients (4.5%) have received transplantation or therapy elsewhere, and 158 patients (51%) have discontinued ibrutinib. Among the 158 patients who have discontinued ibrutinib, 83 patients (52.5%) discontinued as a result of disease progression, classified as transformation (including Richter transformation [n=27] and prolymphocytic leukemia [n=1]) or progressive CLL (n=55). An additional 75 patients (24%) discontinued treatment as a result of adverse events, including 31 patients with infection. Of the patients with infection, 21 died within 30 days of the infection. For these patients with fatal infections, 12 had pneumonia, seven had unspecified sepsis, and two had Pseudomonas bacteremia. The median time on ibrutinib before discontinuation was 98 days. Transformation tended to occur within the first 2 years of therapy and CLL progressions tended to occur later, with an estimated cumulative incidence of CLL progression at 1 year of 0.7% (95% CI, 0% to 1.6%), which increased to an estimated incidence of 19.1% (95% CI, 13.9% to 24.3%) at 4 years (FIG. 2); discontinuation for adverse reasons other than transformation or CLL progression seemed to reach a plateau after 3 years (FIG. 2).

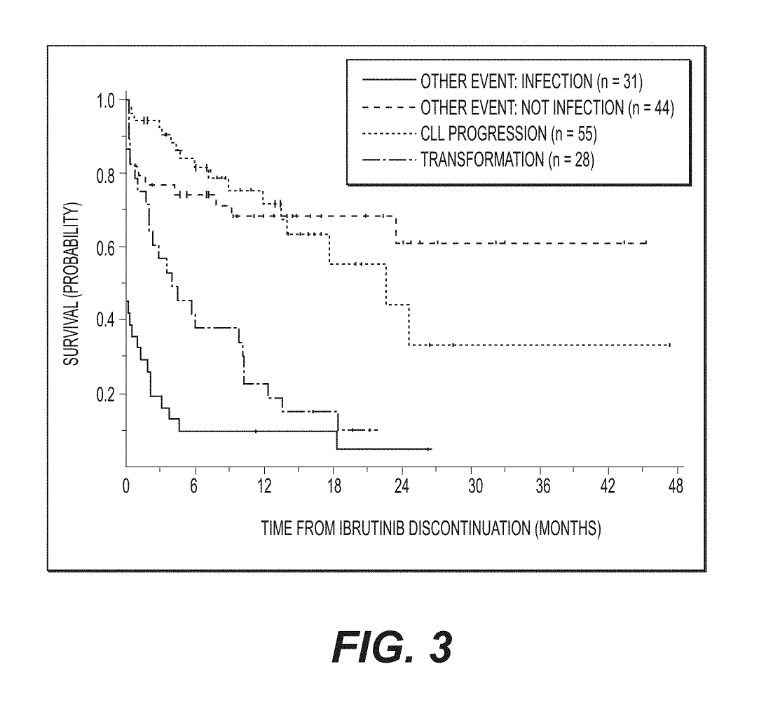

Baseline risk factors for transformation, CLL progression, and other adverse reasons at an earlier follow-up, but with longer follow-up, stronger conclusions can be made. Of the variables considered, complex karyotype at baseline was the variable most strongly associated with risk for transformation; although the variable of MYC abnormalities was the next strongest variable associated with risk for transformation, it did not reach statistical significance (P=0.051; Table 2). Because few CLL progressions occurred before 1 year, the test for proportional hazards was violated for potentially important prognostic variables (eg, age and IGHV status) when using all data across all time points. Therefore, a multivariable model was constructed using a landmark analysis at 1 year. Complex karyotype at baseline, presence of del(17)(p13.1) on fluorescent in situ hybridization, and age less than 65 years were all independently associated with a risk for CLL progression (Table 2). Importantly, for patients age $65 years without complex karyotype or del(17)(p13.1) (n=52), the risk of CLL progression was extremely low, with an estimated cumulative incidence of progression of 1.9% (95% CI, 0% to 5.7%) at 4 years. Conversely, for patients younger than age 65 with both complex karyotype and del(17)(p13.1) (n=46), the estimated cumulative incidence of progression at 4 years was 44% (95% CI, 26% to 61%). Independent risk factors for ibrutinib discontinuation for adverse reasons other than transformation or CLL progression include older age and higher number of prior therapies (Table 2). Median survival time from ibrutinib discontinuation was 3.9 months (95% CI, 2.0 to 10.1 months) for patients with transformation and 22.7 months (95% CI, 13.5 months to not reached; FIG. 3) for patients with CLL progression.

TABLE 2
Multivariable Models for Cumulative Incidence of Ibrutinib
Discontinuation According to Reason for Discontinuation

| | Transformation | | Progressive CLL* | | Other Event |
|---|---|---|---|---|---|
| | HR (95% | | HR (95% | | HR (95% |
| Variable | Cl) | | Cl) | | Cl) |
| Complex | 5.00 (1.51 | | 2.81 (1.34 | | — |
| karyptype (yes | to 16.52) | 008 | to 5.88) | 006 | |
| v no) | | | | | |

| | | | | |
|---|---|---|---|---|
| MYC | 2.15 (1.00 | 051 | — | — |
| abnormality | to 4.65) | | | |
| (yes v no) | | | | |

| del(17)(p13.1) | — | 2.14 (1.15 | 016 | — | |
|---|---|---|---|---|---|
| present on | | to 3.96) | | | |
| FISH (yes | | | | | |
| v no) | | | | | |
| Age | — | 0.49 (0.27 | 023 | 2.02 (1.25 | |
| (≥v<65 years) | | to 0.91) | | to 3.28) | 004 |

| | | | | |
|---|---|---|---|---|
| Prior | — | — | 1.99 (1.23 | 005 |
| therapies >3 | | | to 3.23) | |
| (yes v no) | | | | |

NOTE.
All models were adjusted for treatment with ibrutinib monotherapy versus combination therapy with ibrutinib and ofatumumab.
Abbreviations:
CLL, chronic lymphocytic leukemia;
FISH, fluorescent in situ hybridization;
HR, hazard ratio.
*Landmark analysis at 1 year.

CLL Progression on Ibrutinib is Associated with Acquired Mutations in BTK or PLCG2, which can be Detected Before Relapse

Forty-six patients with progressive CLL had samples at relapse available for deep sequencing by Ion Torrent. Of these, 40 patients (87%; 95% CI, 74% to 95%) had mutations in BTK and/or PLCG2 at the time of clinical relapse. Distribution of mutations included patients with BTK C481 mutation only (n=31), mutation in PLCG2 at described hotspots only (n=3), and mutations in both BTK and PLCG2 (n=6). Variant allele frequency was varied and generally correlated with the presence of disease progression in the peripheral blood versus primarily nodal relapse. For the seven patients with variant allele frequency of less than 10%, all but one had disease progression only in the lymph nodes, with low lymphocyte count in the peripheral blood.

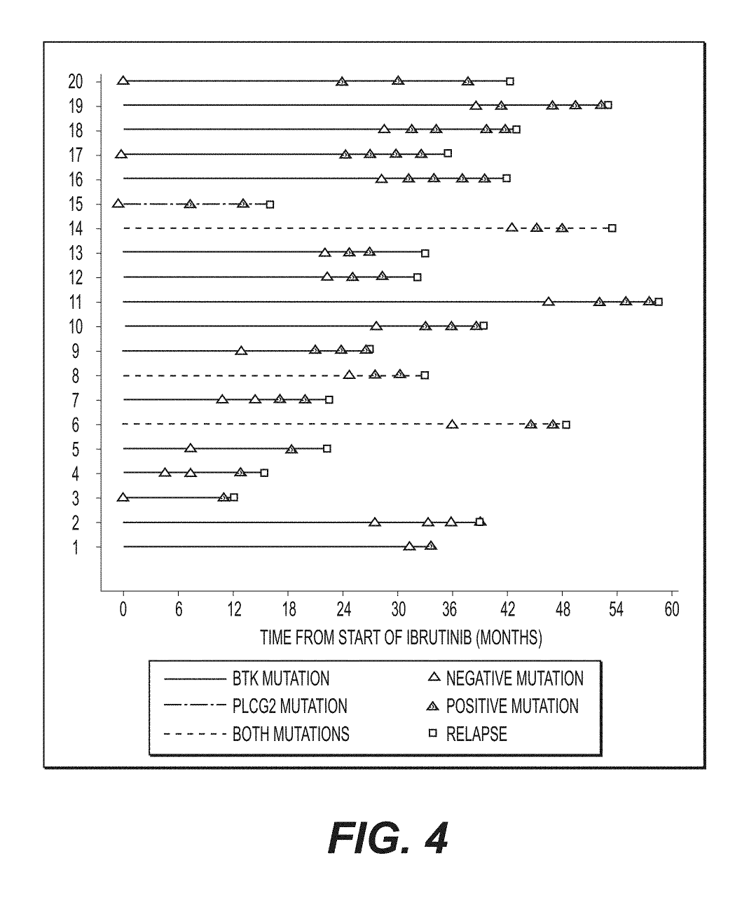

For 20 patients who had acquired BTK or PLCG2 mutations, serial samples were available before relapse and were analyzed retrospectively for mutations. Among these patients, relapse was detected incidentally in two patients, by bone marrow biopsy in one patient, by increasing lymphocytosis alone in four patients, by lymph node enlargement alone in eight patients, and by both lymphocytosis and enlarging nodes in five patients. A clone of resistant cells can first be detected a median of 9.3 months before clinical relapse (95% CI, 7.6 to 11.7 months; FIG. 4), occurring as close as 3 months or greater than 18 months before clinical relapse.

Clones of Ibrutinib-Resistant Cells can be Detected Prospectively Before Clinical Relapse

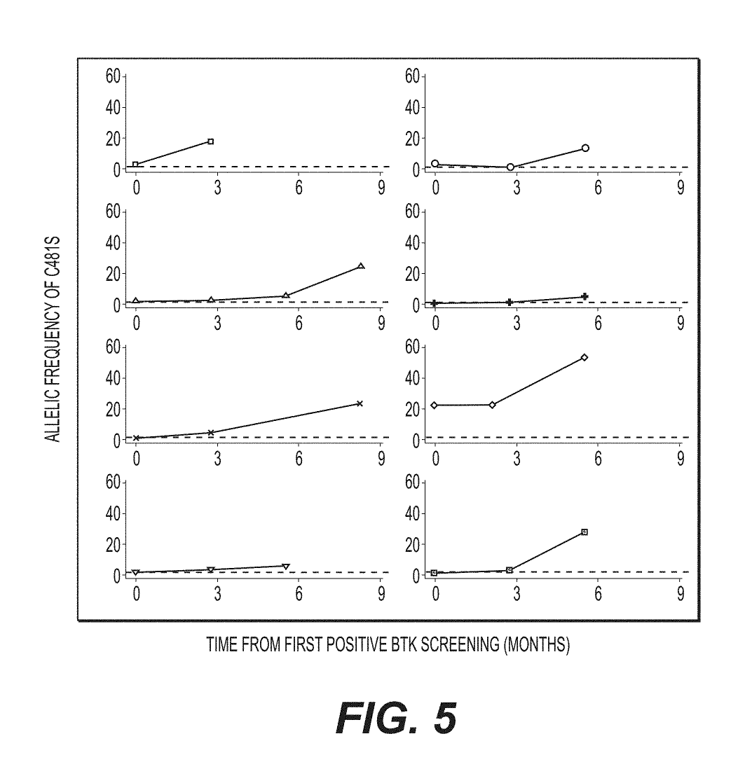

On the basis of the finding that patients who experienced relapse on ibrutinib often had rapid progression and poor outcomes, a clinical-grade mutation monitoring strategy was initiated in the institutional Clinical Laboratory Improvement Amendments-certified molecular laboratory starting in November 2014. Mutational analysis of the entire coding regions of BTK and PLCG2 was performed on a cohort of 112 patients every 3 months prospectively. To date, eight patients have experienced clinical relapse, and all eight patients had BTK^{C481S} mutations with expansion of the clone before relapse. BTK^{C481S} mutations of greater than 1% allelic frequency were detected in an additional eight patients. All but one patient who discontinued therapy and went to hospice without clinical relapse have had increasing circulating CLL cells in the peripheral blood by flow cytometry, and all have had expansion of the resistant clones (FIG. 5). Four of these seven patients have increasing lymph node size on computed tomography scan, but none have yet met criteria for clinical relapse. No patients in this cohort have experienced clinical relapse, and no early signs of progression have been noted in patients without BTK^{C481S} clones.

### DISCUSSION

Identified herein are mutations in BTK and PLCG2 as one mechanism of resistance to ibrutinib in CLL. With a large cohort of patients, it is definitively demonstrated herein that these mutations are the predominant mechanism by which CLL becomes resistant to ibrutinib. Perhaps most significantly, it is shown that clinical resistance is preceded by a prolonged period of asymptomatic clonal expansion, which indicates the ability to pre-emptively target these cells with alternative therapies in the context of a clinical trial before the patient becomes acutely ill with refractory disease. Particularly for high-risk patients in whom allogeneic stem-cell transplantation is often considered and debated as appropriate, such monitoring offers the opportunity to transition these patients to this modality when early signs of resistance are identified. Although data regarding the best approach to these patients are not yet available, this is certainly an area of active investigation.

Both complex karyotype and del(17) (p13.1) are associated with disease progression on ibrutinib, indicating that markers that predict poor prognosis with standard therapies also confer risk with novel therapies, albeit a delayed risk. Notably, age is an important risk factor, with younger patients being more likely to experience relapse than older patients. Younger patients may have a different disease biology that predisposes them to relapse, or alternatively, differences in drug metabolism with aging may allow higher drug exposures and therefore higher BTK occupancy in older patients, making them less likely to develop resistance mutations. In addition, biochemical properties of the tumor cells may be different based on age.

The data show that relapse can occur with varied allelic frequencies of mutant cells in the peripheral blood. In the series, low allelic frequencies were generally associated with progression in the lymph nodes only without corresponding peripheral-blood progression. This indicates that progression can be associated with a compartmental acquisition of mutations, with the bulk of the mutated cells present in the lymph node compartment. In addition, mutations at low allelic frequencies in the presence of peripheral-blood disease indicate that cooperating mutations are important, and further studies into the clonal dynamics of progression are warranted. Because relapse on ibrutinib can cause an accelerated-phase disease, especially when ibrutinib is discontinued, the finding that a period of asymptomatic mutant clonal expansion precedes relapse is of critical importance. Shown herein is that targeted sequencing for these mutations in the peripheral blood is feasible and can be an effective biomarker to predict relapse in asymptomatic patients. One therapeutic option that is currently under active investigation is adding a second agent to ibrutinib as soon as a mutant clone is detected to prevent clonal expansion. In addition, in select patients with an identified donor, this period of asymptomatic clonal expansion can allow for stem-cell transplantation, although additional cytoreductive therapy before transplantation may be needed.

These data show that ibrutinib resistance in patients with high-risk, previously treated CLL is a problem of increasing clinical significance with potentially devastating consequences. Because ibrutinib has been approved by the US Food and Drug Administration for use in the relapsed setting for 2 years, the first emergence of relapse in the community setting is just starting to be observed. Enhanced knowledge of both the molecular and clinical mechanisms of relapse allows for strategic alterations in monitoring and management that can change the natural history of ibrutinib resistance.

SEQUENCES

Bruton Tyrosine Kinase (BTK) amino acid sequence (GenBank
Accession No. AAB60639.1)
SEQ ID NO: 1

| | |
|---|---|
| 1 | maavilesif lkrsqqkkkt splnfkkrlf lltvhklsyy eydfergrrg skkgsidvek |
| 61 | itcvetvvpe knppperqip rrgeesseme qisiierfpy pfqvvydegp lyvfspteel |
| 121 | rkrwihqlkn virynsdlvq kyhpcfwidg qylccsqtak namgcqilen rngslkpgss |
| 181 | hrktkkplpp tpeedqilkk plppepaaap vstselkkvv alydympmna ndlglrkgde |
| 241 | yfileesnlp wwrardkngq egyipsnyvt eaedsiemye wyskhmtrsq aegllkqegk |
| 301 | eggfivrdss kagkytvsvf akstgdpqgv irhyvvcstp qsqyylaekh lfstipelin |
| 361 | yhqhnsagli srlkypvsqq nknapstagl gygsweidpk dltflkelgt gqfgvvkygk |
| 421 | wrgqydvaik mikegsmsed efieeakvmm nlsheklvql ygvctkqrpi fiiteymang |
| 481 | cllnylremr hrfqtqqlle mckdvceame yleskqflhr dlaarnclvn dqgvvkvsdf |
| 541 | glsryvldde ytssvgskfp vrwsppevlm yskfssksdi wafgvimwei yslgkmpyer |
| 601 | ftnsetaehi agglrlyrph lasekvytim yscwhekade rptfkillsn ildvmdees |

Bruton Tyrosine Kinase (BTK) nucleic acid sequence (GenBank
Accession No. NM_000061.2)
SEQ ID NO: 2

| | |
|---|---|
| 1 | aactgagtgg ctgtgaaagg gtggggtttg ctcagactgt ccttcctctc tggactgtaa |
| 61 | gaatatgtct ccagggccag tgtctgctgc gatcgagtcc caccttccaa gtcctggcat |
| 121 | ctcaatgcat ctgggaagct acctgcatta agtcaggact gagcacacag gtgaactcca |
| 181 | gaaagaagaa gctatggccg cagtgattct ggagagcatc tttctgaagc gatcccaaca |
| 241 | gaaaaagaaa acatcacctc taaacttcaa gaagcgcctg tttctcttga ccgtgcacaa |
| 301 | actctcctac tatgagtatg actttgaacg tgggagaaga ggcagtaaga agggttcaat |
| 361 | agatgttgag aagatcactt gtgttgaaac agtggttcct gaaaaaaatc ctcctccaga |
| 421 | aagacagatt ccgagaagag gtgaagagtc cagtgaaatg gagcaaattt caatcattga |
| 481 | aaggttccct tatcccttcc aggttgtata tgatgaaggg cctctctacg tcttctcccc |
| 541 | aactgaagaa ctaaggaagc ggtggattca ccagctcaaa aacgtaatcc ggtacaacag |
| 601 | tgatctggtt cagaaatatc acccttgctt ctggatcgat gggcagtatc tctgctgctc |
| 661 | tcagacagcc aaaaatgcta tgggctgcca aattttggag aacaggaatg gaagcttaaa |
| 721 | acctgggagt tctcaccgga agacaaaaaa gcctcttccc ccaacgcctg aggaggacca |
| 781 | gatcttgaaa aagccactac cgcctgagcc agcagcagca ccagtctcca caagtgagct |
| 841 | gaaaaaggtt gtggcccttt atgattacat gccaatgaat gcaaatgatc tacagctgcg |
| 901 | gaagggtgat gaatatttta tcttggagga aagcaactta ccatggtgga gagcacgaga |
| 961 | taaaaatggg caggaaggct acattcctag taactatgtc actgaagcag aagactccat |
| 1021 | agaaatgtat gagtggtatt ccaaacacat gactcggagt caggctgagc aactgctaaa |
| 1081 | gcaagagggg aaagaaggag gtttcattgt cagagactcc agcaaagctg gcaaatatac |
| 1141 | agtgtctgtg tttgctaaat ccacagggga ccctcaaggg gtgatacgtc attatgttgt |
| 1201 | gtgttccaca cctcagagcc agtattacct ggctgagaag caccttttca gcaccatccc |
| 1261 | tgagctcatt aactaccatc agcacaactc tgcaggactc atatccaggc tcaaatatcc |
| 1321 | agtgtctcaa caaaacaaga atgcaccttc cactgcaggc ctgggatacg gatcatggga |
| 1381 | aattgatcca aaggacctga ccttcttgaa ggagctgggg actggacaat ttggggtagt |
| 1441 | gaagtatggg aaatggagag gccagtacga cgtggccatc aagatgatca aagaaggctc |
| 1501 | catgtctgaa gatgaattca ttgaagaagc caaagtcatg atgaatcttt cccatgagaa |
| 1561 | gctggtgcag ttgtatggcg tctgcaccaa gcagcgcccc atcttcatca tcactgagta |
| 1621 | catggccaat ggctgcctcc tgaactacct gagggagatg cgccaccgct tccagactca |
| 1681 | gcagctgcta gagatgtgca aggatgtctg tgaagccatg gaatacctgg agtcaaagca |
| 1741 | gttccttcac cgagacctgg cagctcgaaa ctgtttggta aacgatcaag gagttgttaa |
| 1801 | agtatctgat ttcggcctgt ccaggtatgt cctggatgat gaatacacaa gctcagtagg |
| 1861 | ctccaaattt ccagtccggt ggtccccacc ggaagtcctg atgtatagca agttcagcag |
| 1921 | caaatctgac atttgggctt ttggggtttt gatgtgggaa atttactccc tggggaagat |
| 1981 | gccatatgag agatttacta acagtgagac tgctgaacac attgcccaag gcctacgtct |
| 2041 | ctacaggcct catctggctt cagagaaggt atataccatc atgtacagtt gctggcatga |
| 2101 | gaaagcagat gagcgtccca ctttcaaaat tcttctgagc aatattctag atgtcatgga |
| 2161 | tgaagaatcc tgagctcgcc aataagcttc ttggttctac ttctcttctc cacaagcccc |
| 2221 | aatttcactt tctcagagga aatcccaagc ttaggagccc tggagccttt gtgctcccac |
| 2281 | tcaatacaaa aaggcccctc tctacatctg ggaatgcacc tcttctttga ttccctggga |
| 2341 | tagtggcttc tgagcaaagg ccaagaaatt attgtgcctg aaatttcccg agagaattaa |
| 2401 | gacagactga atttgcgatg aaaatatttt ttaggaggga ggatgtaaat agccgcacaa |
| 2461 | aggggtccaa cagctctttg agtaggcatt tggtagagct tgggggtgtg tgtgtggggg |
| 2521 | tggaccgaat ttggcaagaa tgaaatggtg tcataaagat gggaggggag ggtgttttga |
| 2581 | taaaataaaa ttactagaaa gcttgaaagt c |

### REFERENCES

Burger J A, Tedeschi A, Barr P M, et al: Ibrutinib as initial therapy for patients with chronic lymphocytic leukemia. N Engl J Med 373:2425-2437, 2015
Byrd J C, Brown J R, O'Brien S, et al: Ibrutinib versus ofatumumab in previously treated chronic lymphoid leukemia. N Engl J Med 371:213-223, 2014
Byrd J C, Furman R R, Coutre S E, et al: Targeting BTK with ibrutinib in relapsed chronic lymphocytic leukemia. N Engl J Med 369:32-42, 2013
Cheng S, Guo A, Lu P, et al: Functional characterization of BTK(C481S) mutation that confers ibrutinib resistance: Exploration of alternative kinase inhibitors. Leukemia 29:895-900, 2015
Famà R, Bomben R, Rasi S, Dal Bo M, Ciardullo C, Monti S, Rossi F, D'Agaro T, Zucchetto A, Gattei V, Gaidano G. Ibrutinib-naïve chronic lymphocytic leukemia lacks Bruton tyrosine kinase mutations associated with treatment resistance. Blood. 2014 Dec. 11; 124(25):3831-3.
Fowler N, Sharman J P, Smith S M, et al: The Btk inhibitor, PCI-32765, induces durable responses with minimal toxicity in patients with relapsed/refractory B-cell malignancies: Results from a phase I study. Blood 116:964, 2010 (abstr)
Furman R R, Cheng S, Lu P, et al: Ibrutinib resistance in chronic lymphocytic leukemia. N Engl J Med 370:2352-2354, 2014
Herman S E, Gordon A L, Hertlein E, et al: Bruton tyrosine kinase represents a promising therapeutic target for treatment of chronic lymphocytic leukemia and is effectively targeted by PCI-32765. Blood 117:6287-6296, 2011
Herman S E, Mustafa R Z, Gyamfi J A, et al: Ibrutinib inhibits BCR and NF-kB signaling and reduces tumor proliferation in tissue-resident cells of patients with CLL. Blood 123:3286-3295, 2014
Jaglowski S M, Jones J A, Nagar V, et al: Safety and activity of BTK inhibitor ibrutinib combined with ofatumumab in chronic lymphocytic leukemia: A phase 1b/2 study. Blood 126:842-850, 2015
Liu T M, Woyach J A, Zhong Y, et al: Hypermorphic mutation of phospholipase C, g2 acquired in ibrutinib-resistant CLL confers BTK independency upon B-cell receptor activation. Blood 126:61-68, 2015
Maddocks K, Flynn J M, Andritsos L, et al: A phase II study of the BTK inhibitor ibrutinib in genetic risk-stratified relapsed and refractory patients with chronic lymphocytic leukemia (CLL)/small lymphocytic lymphoma (SLL). European Hematology Association Annual Meeting, Milan, Italy, Jun. 12-15, 2014 (abstr S1343)
Maddocks K J, Ruppert A S, Lozanski G, et al: Etiology of ibrutinib therapy discontinuation and outcomes in patients with chronic lymphocytic leukemia. JAMA Oncol 1:80-87, 2015
Thompson P A, O'Brien S M, Wierda W G, et al: Complex karyotype is a stronger predictor than del (17p) for an inferior outcome in relapsed or refractory chronic lymphocytic leukemia patients treated with ibrutinib-based regimens. Cancer 121:3612-3621, 2015
Woyach J A, Bojnik E, Ruppert A S, et al: Bruton's tyrosine kinase (BTK) function is important to the development and expansion of chronic lymphocytic leukemia (CLL). Blood 123:1207-1213, 2014
Woyach J A, Furman R R, Liu T M, et al. Resistance mechanisms for the Bruton's tyrosine kinase inhibitor ibrutinib. N Engl J Med 370:2286-2294, 2014
Woyach J A, Ruppert A S, Lozanski G, Lozanski A, Heerema N A, Zhao W, Abruzzo L, Gordon A, Jones J A, Flynn J M, Jaglowski S M. Association of disease progression on ibrutinib therapy with the acquisition of resistance mutations: A single-center experience of 267 patients. In ASCO Annual Meeting Proceedings 2014 May 20 (Vol. 32, No. 15 suppl, p. 7010).
Young R M, Staudt L M. Ibrutinib treatment of CLL: the cancer fights back. Cancer cell. 2014 Jul. 14; 26(1): 11-3.

What is claimed is:

1. A method for detecting a Bruton agammaglobulinemia tyrosine kinase (BTK) mutation in B cells, comprising assaying DNA and RNA in B cells for one or more BTK mutations by polymerase chain reaction PCR, comparing the number of DNA and RNA mutations in each cell to identify a mutation:wildtype ratio, thereby determining both the percentage of mutated cells in the sample and the level of expression of the mutated BTK molecules in each cell.

2. The method of claim 1, wherein the PCR reaction is selected from the group consisting of digital droplet PCR (ddPCR), digital droplet qualitative PCR (ddQPCR), reverse transcriptase PCR (RT-PCR), and real-time PCR.

3. The method of claim 1, wherein the RNA is assayed by converting it first to cDNA using a reverse transcriptase.

4. The method of claim 1, wherein the BTK mutation comprises a mutation at C481 as set forth in SEQ ID NO: 1.

5. The method of claim 4, wherein the BTK mutation comprises a C481S, C481R, C481F, or C481V mutation.

6. The method of claim 1, wherein the BTK mutation comprises one or more mutations at G1442 and T1441 as set forth in SEQ ID NO: 2.

7. The method of claim 6, wherein the one or more BTK mutation comprises a T1441A, T1441C, T1441G, G1442C, G1442, or G1442A mutation.

8. A method for detecting resistance to a BTK inhibitor in a subject with chronic lymphocytic leukemia (CLL), comprising assaying DNA and RNA in B cells from the subject for one or more BTK mutations by PCR, comparing the number of DNA and RNA mutations in each cell to identify a mutation:wildtype ratio, wherein an increase in the percentage of cells comprising a BTK mutation in the sample, an increase in the level of expression of the mutated BTK molecules in one or more cells, or a combination thereof indicates that the subject has a cancer that is resistant to BTK inhibitors.

9. The method of claim 8, wherein the BTK inhibitor comprises ibrutinib.

10. The method of claim 1, wherein the PCR reaction is selected from the group consisting of digital droplet PCR (ddPCR), digital droplet qualitative PCR (ddQPCR), reverse transcriptase PCR (RT-PCR), and real-time PCR.

11. The method of claim 8, wherein the RNA is assayed by converting it first to cDNA using a reverse transcriptase.

12. The method of claim 8, wherein the BTK mutation comprises a mutation at C481 as set forth in SEQ ID NO: 1.

13. The method of claim 12, wherein the BTK mutation comprises a C481S, C481R, C481F, or C481V mutation.

14. The method of claim 8, wherein the BTK mutation comprises one or more mutation at G1442 or T1441 as set forth in SEQ ID NO: 2.

15. The method of claim 14, wherein the one or more BTK mutations comprises a T1441A, T1441C, T1441G, G1442C, G1442, or G1442A mutation.

16. A method of treating chronic lymphocytic leukemia (CLL) comprising assaying nucleic acid in B cells from the subject for one or more BTK mutations by PCR, comparing the number of DNA and RNA mutations in each cell to identify a mutation:wildtype ratio, wherein an increase in the percentage of cells comprising a BTK mutation in the sample, an increase in the level of expression of the mutated BTK molecules in one or more cells, or a combination thereof indicates that the subject has a cancer that is resistant to BTK inhibitors; wherein in a subject with a BTK inhibitor resistant cancer the method further comprises administering to the subject a cancer therapeutic that is not a BTK inhibitor; and wherein in a subject without a BTK inhibitor resistant cancer the method comprises administering to the subject a BTK inhibitor.

17. The method of claim 16, wherein the PCR reaction is selected from the group consisting of digital droplet PCR (ddPCR), digital droplet qualitative PCR (ddQPCR), reverse transcriptase PCR (RT-PCR), and real-time PCR.

18. The method of claim 16, wherein the BTK mutation comprises a mutation at C481 as set forth in SEQ ID NO: 1.

19. The method of claim 18, wherein the BTK mutation comprises a C481S, C481R, C481F, or C481V mutation.

20. The method of claim 16, wherein the BTK mutation comprises one or more mutation at G1442 or T1441 as set forth in SEQ ID NO: 2.

21. The method of claim 20, wherein the one or more BTK mutations comprises a T1441A, T1441C, T1441G, G1442C, G1442, or G1442A mutation.

22. The method of claim 16, wherein the BTK inhibitor comprises Ibrutinib, Acalabrutinib, ONO/GS-4059, Spebrutnib, HM71224, or BGB-3111.
